(19) United States
(12) Patent Application Publication
(10) Pub. No.: US 2026/0115413 A1
(43) Pub. Date: Apr. 30, 2026

(54) CATHETER STEERING MECHANISM

(71) Applicant: Boston Scientific Scimed, Inc., Maple Grove, MN (US)

(72) Inventors: Brian Ma, Etobicoke (CA); Miles Montgomery, East Gwillimbury (CA); Jean-Olivier Normandeau, Toronto (CA); Yara Hosein, Mississauga (CA); Ian Major, Acton (CA); Kevin Martin, Rogers, MN (US)

(21) Appl. No.: 19/373,218

(22) Filed: Oct. 29, 2025

Related U.S. Application Data
(60) Provisional application No. 63/714,008, filed on Oct. 30, 2024.

(51) Int. Cl. A61M 25/00; A61M 25/01

(52) U.S. Cl. CPC A61M 25/001; A61M 25/0045; A61M 25/005; A61M 2025/015

(57) ABSTRACT

A catheter comprising an elongated shaft defining a lumen and having a distal portion is disclosed. The elongated shaft includes an elongated support member coaxial with the lumen. The support member has a distal end. A pull ring assembly including a pull ring and a pull wire. The pull ring is disposed on the shaft at the distal portion, and the pull wire is attached to the pull ring and extends proximally along the shaft. The pull ring includes an outer surface, a pull ring distal end, and a pull ring proximal end. The support member is attached to the outer surface of the pull ring such that the distal end of the support member is disposed distal to the pull ring proximal end.

### CROSS REFERENCE TO RELATED APPLICATIONS

The present application claims priority to U.S. patent application Ser. No. 63/714,008, filed Oct. 30, 2024, the disclosure of which is incorporated herein in its entirety.

### TECHNICAL FIELD

The present disclosure relates to medical devices for catheterization procedures, such as medical devices for electrophysiological procedures. More specifically, the present disclosure relates to steerable catheters and methods for manufacturing steerable catheters.

### BACKGROUND

Various medical fields use different types of catheters to achieve access to a physiological site in medical procedures. For instance, electrophysiological procedures involve guiding catheters into the heart and tracking the location of the catheters with respect to the heart. Catheter ablation is minimally invasive electrophysiological procedure to treat a variety of heart conditions such as supraventricular and ventricular arrhythmia. Example catheters used in catheter ablation can include mapping catheters, ablation catheters, guiding sheaths or introducer sheaths, dilators, and other medical tools, which can be referred to as catheters in this disclosure.

To position a catheter within the body at a target site, some type of navigation mechanism is used, such as a steering feature incorporated into the catheter. Clinicians manually manipulate or operate the catheter using the steering feature. To facilitate the advancement of catheters through a patient's vasculature, the concurrent application of torque at a proximal portion of the catheter and the ability to deflect the distal portion of the catheter in a selected direction permits the clinician to adjust the direction of advancement of the distal portion of the catheter and to selectively position the distal portion of the catheter during an electrophysiological procedure. In an example, the distal portion includes a pull ring assembly having a pull ring that is deflected with a pull wire or other tension member attached or anchored at the distal end of the catheter and extending proximally to an actuator in a control handle that controls the application of tension on the pull wire. Often, a catheter is configured to deflect in more than one direction and includes one or more pull wires installed on the pull ring on opposite side from each other. For example, the catheter can include a pair of pull wires or two pairs of pull wires for four-way steering.

### SUMMARY

In Example 1, a catheter, comprising: an elongated shaft defining a lumen and having a distal portion, the elongated shaft having an elongated support member coaxial with the lumen, the support member having a distal end; and a pull ring assembly including a pull ring disposed on the shaft at the distal portion and a pull wire attached to the pull ring and extending proximally along the shaft, the pull ring having an outer surface, a pull ring distal end, and a pull ring proximal end; wherein the support member is attached to the outer surface of the pull ring such that the distal end of the support member is disposed distal to the pull ring proximal end.

In Example 2, the catheter of Example 1, wherein the support member is attached to the outer surface of the pull ring such that the distal end of the support member is disposed between the pull ring distal and proximal ends.

In Example 3, the catheter of any of Examples 1 and 2, wherein the support member includes a braided material forming a braided member or a coiled material forming a coiled member.

In Example 4, the catheter of any of Examples 1-3, wherein the support member is an inner most support member.

In Example 5, the catheter of any of Examples 1-4, wherein the shaft further includes an elongate liner layer in the distal portion defining an inner wall of the lumen and having a liner layer outer surface, wherein the support member is radially disposed on the liner layer outer surface.

In Example 6, the catheter of Example 5, wherein the pull ring is radially disposed between the liner layer and the support member.

In Example 7, the catheter of any of Examples 1-6, wherein the shaft includes a cover disposed on the pull ring assembly and support member.

In Example 8, the catheter of any of Examples 5-7, wherein the liner layer extends distally from the pull ring.

In Example 9, the catheter of Example 8, wherein the cover is disposed directly onto the liner layer outer surface distal to the pull ring.

In Example 10, the catheter of any of Examples 1-9, wherein the support member is welded or bonded to the outer surface of the pull ring.

In Example 11, the catheter of any of Examples 1-10, and further comprising a compression member to attach the support member against the pull ring.

In Example 12, the catheter of Example 11, wherein the compression member encircles the support member and the pull ring to hold the support member in place.

In Example 13, the catheter of Example 12, wherein the compression member is a sleeve of a heat-shrink material or metallic material.

In Example 14, the catheter of any of Examples 1-13, wherein the shaft includes a proximal end coupled to a handle, wherein the handle includes an actuator coupled to the pull wire.

In Example 15, the catheter of any of Examples 1-14, wherein the catheter is a guide catheter.

In Example 16, a catheter, comprising: an elongated shaft defining a lumen and having a distal portion, the elongated shaft having an elongated support member coaxial with the lumen, the support member having a distal end; and a pull ring assembly including a pull ring disposed on the shaft at the distal portion and a pull wire attached to the pull ring and extending proximally along the shaft, the pull ring having an outer surface, a pull ring distal end, and a pull ring proximal end; wherein the support member is attached to the outer surface of the pull ring such that the distal end of the support member is disposed distal to the pull ring proximal end.

In Example 17, the catheter of Example 16, wherein the support member is attached to the outer surface of the pull ring such that the distal end of the support member is disposed between the pull ring distal and proximal ends.

In Example 18, the catheter of Example 16, wherein the support member includes a braided material forming a braided member or a coiled material forming a coiled member.

In Example 19, the catheter of Example 16, wherein the distal end of the support member is disposed distal to the pull ring distal end.

In Example 20, the catheter of Example 16, wherein the shaft includes one support member.

In Example 21, the catheter of Example 16, wherein the shaft further includes an elongate liner layer in the distal portion defining an inner wall of the lumen and having a liner layer outer surface, wherein the support member is radially disposed on the liner layer outer surface.

In Example 22, the catheter of Example 21, wherein the pull ring is radially disposed between the liner layer and the support member.

In Example 23, the catheter of Example 21, wherein the liner layer extends distally from the pull ring.

In Example 24, the catheter of Example 23, wherein the shaft includes a cover disposed on the pull ring assembly and support member.

In Example 25, the catheter of Example 24, wherein the cover is disposed directly onto the liner layer outer surface distal to the pull ring.

In Example 26, the catheter of Example 16, wherein the support member is welded to the outer surface of the pull ring.

In Example 27, the catheter of Example 16, wherein the support member is attached to the outer surface of the pull ring via a compression member.

In Example 28, the catheter of Example 27, wherein the compression member encircles the support member and the pull ring.

In Example 29, the catheter of Example 28, wherein the compression member is a sleeve of a heat-shrink material.

In Example 30, a catheter comprising: an elongated shaft defining a lumen and having a distal portion, the elongated shaft having an elongated support member coaxial with the lumen, the support member having a distal end; and a pull ring assembly including a pull ring disposed on the shaft at the distal portion and a pull wire attached to the pull ring and extending proximally along the shaft, the pull ring having an outer surface, a pull ring distal end, and a pull ring proximal end; wherein the distal end of the support member is attached to the outer surface of the pull ring.

In Example 31, the catheter of Example 30, wherein the support member includes a braided material forming a braided member or a coiled material forming a coiled member.

In Example 32, the catheter of Example 31, wherein the support member is welded or bonded to the outer surface of the pull ring.

In Example 33, a method of manufacturing a catheter, comprising: providing an elongated shaft defining a lumen and having a distal portion, the elongated shaft having an elongated support member coaxial with the lumen, the support member having a distal end; providing a pull ring assembly including a pull ring disposed on the shaft at the distal portion and a pull wire attached to the pull ring and extending proximally along the shaft, the pull ring having an outer surface, a pull ring distal end, and a pull ring proximal end; and attaching the support member to the outer surface of the pull ring such that the distal end of the support member is disposed between the pull ring distal and proximal ends.

In Example 34, the method of Example 33, wherein attaching the support member to the outer surface of the pull ring includes welding or bonding the support member to the outer surface of the pull ring.

In Example 35, the method of Example 33, where attaching the support member to the outer surface of the pull ring includes encircling the support member and the pull ring with a compression member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2 is a schematic diagram illustrating an example catheter that can be used with the example electrophysiology system of FIG. 1.

FIG. 3 is a schematic diagram of a distal portion of a prior art catheter that can be used with the example electrophysiology system of FIG. 1.

FIG. 4A is a schematic diagram illustrating from a side view an embodiment of the catheter of FIG. 2.

FIG. 4B is a schematic diagram illustrating from an end view the catheter of FIG. 4A as a cross section of FIG. 4A taken along line 4-4.

FIG. 5A is a schematic diagram illustrating from a side view an embodiment of the catheter of FIG. 2.

FIG. 5B is a schematic diagram illustrating from an end view the catheter of FIG. 5A as a cross section of FIG. 5A taken along line 5-5.

FIG. 6 is a block diagram illustrating an example method of manufacturing the catheter of FIG. 2.

FIG. 7 is a schematic diagram illustrating from a side view another embodiment a catheter that can be used with the example electrophysiology system of FIG. 1.

FIG. 8 is a schematic diagram illustrating from a side view another embodiment a catheter that can be used with the example electrophysiology system of FIG. 1.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

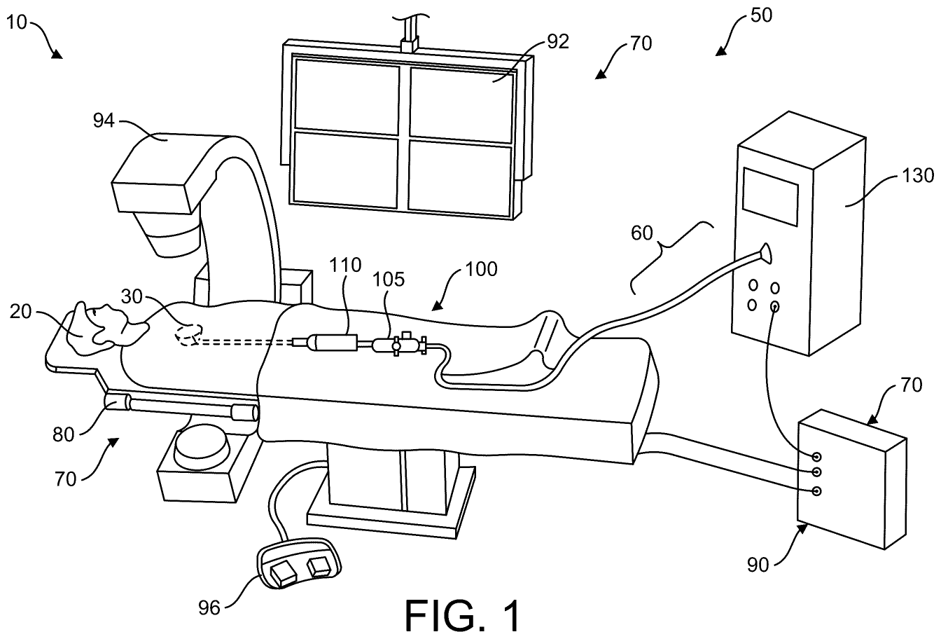

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which in the example includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheters. For instance, a catheter defines a longitudinal axis that passes through a centroid of a cross section of the catheter, such as the centroid of a cross section of a shaft of catheter 105 or a centroid of a cross section of a main lumen of a sheath 110. In the example, the catheter 105 is disposed within the sheath 110. The catheters 105, 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated embodiment, the device tracking is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute tracking electrodes on the tracked catheters that generate outputs received and processed by the mapping and navigation controller 90 to track the location of the various location sensing electrodes within the localization volume. For instance, an impedance tracking methodology can employ the use of a patch electrode (not show) attached to the patient's body, a current or impedance based can be determined between the tracking electrode on the catheter and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters include tracking electrodes disposed on the deflectable portions of the catheter shafts. Multiple tracking electrodes can be employed on the deflectable portions of the catheters for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

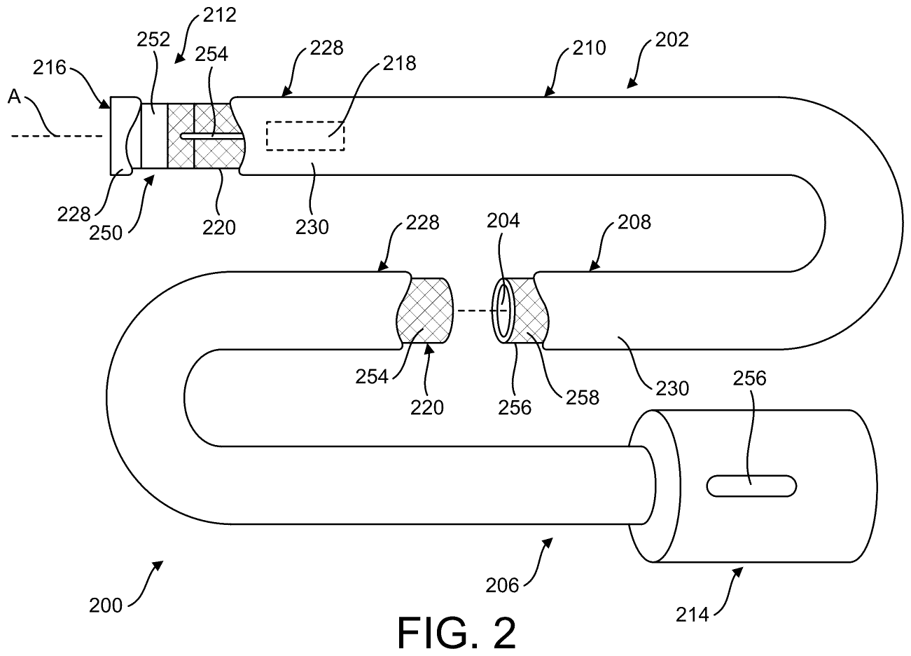

FIG. 2 illustrates an embodiment of a catheter 200 that can be used in the example clinical setting 10 in catheter assembly 100. For instance, catheter 200 can be further configured as a guide catheter, dilator, ablation catheter, or other flexible, deflectable medical tool that can be steered to a target site. The catheter 200 includes an elongated shaft 202, such as an elongated and flexible shaft 202 defining a longitudinal axis A. The shaft 202 also defines a main lumen 204 along the longitudinal axis A and has a proximal portion 206, a longitudinal section 208, and a distal portion 210. The distal portion 210 includes a distal tip section 212 defining a distal tip 216. In some embodiments, the distal tip section 212 can be configured as a dilator tip.

The shaft 202 can be constructed to include several radially disposed layers. An elongated and flexible support member 220 is disposed along some or all of the shaft 202 along the longitudinal axis A. In various embodiments, the support member 220 is a flexible braid such as a braid formed from stainless steel strands, strands of stiff metals such as tungsten, or strands of a high-strength polymer. In some embodiments, the shaft 202 includes a liner layer (not shown in FIG. 2) radially underneath the support member 220 and coaxial with the main lumen 204. The liner layer can define an inner wall of the main lumen 204. In examples such as in which the catheter 200 is a guide sheath or dilator, the liner layer can be a thin wall constructed of polytetrafluoroethylene (PTFE). The shaft 202 further includes a cover member 228 disposed radially over the braided support member 220 to form an outer surface 230 of the shaft 202.

In some embodiments, the shaft 202 includes several components disposed on the distal portion 210. In the illustrated embodiment, the distal portion 210 of the shaft 202 can include tracking devices 218 such as a magnetic tracking device or tracking electrodes, and each tracking device 218 is coupled to a corresponding lead electrical conductor (not shown) extending longitudinally along the shaft 202 and terminated at the proximal portion 206 to carry an electrical signal associated with the tracking device 218. In the example, the magnetic tracking device and tracking electrodes are configured for use with a magnetic-based tracking system and an impedance-based tracking system, respectively, to detect the position of the catheter. In some embodiments, the distal portion includes radiopaque markers, which can be formed from the tracking electrodes in some embodiments. In some embodiments, other components disposed on the distal portion include sensors such as pressure sensors or thermistors, which are also coupled to corresponding lead electrical conductors.

In embodiments, the proximal portion 206 is coupled to a handle 214 proximal to the shaft 202. The handle 214 can include an electrical connection that can be coupled to a tracking system, such as EAM system 70. In one example, the electrical connection is a multipin connector. In some embodiments, such as in the case of the catheter 200 configured as a guide sheath or dilator, the proximal portion 206 or handle 214 can include a hemostatic valve that can be coupled to a source of irrigation fluid and a port to receive other catheters or guidewires within the main lumen 204. In some embodiments, such as in the case of the catheter 200 configured as an ablation catheter, the handle includes electrical connections to be coupled to a source of ablation energy, such as an electrical signal from console 130 or connections to conduits to carry a cryogenic fluid. For instance, the main lumen 204 can be configured to carry other electrical leads, such as leads to the ablation electrodes or other sensors, or a conduit of irrigation fluid along the shaft 202 to the distal portion 210. In some examples, the distal tip section 212 can be configured to include an ablation electrode assembly.

The distal tip section 212 includes a pull ring assembly 250. The pull ring assembly 250 includes a pull ring 252 and a pull wire 254 attached to the pull ring 252. In some embodiments, the pull ring assembly 250 includes one to four pull wires 254 that are attached to a face of the pull ring 252. In one particular example, two diametrically opposed pull wires 254 are attached to the face of the pull ring 252. The pull wires in some examples are not evenly spaced apart. The pull wires 254 extend along the shaft 202 to the proximal portion 206 and are coupled to an actuator 256 on the handle 214. In some embodiments, the pull wires 254 are disposed between the liner layer and the support member 220, such as radially underneath the braided support member 220, along the shaft 202 to the handle 214. Pulling or releasing the pull wires 254 via the actuator 256 during use of the catheter 200 will cause the pull ring 252 to tilt or rock and bend the distal portion 210 of the catheter shaft 202 and affect steering of the catheter 200. The pull ring 252 is fixed in relation to the shaft 202, and the pull ring does not translate longitudinally with respect to the shaft 202.

The support member 220 is illustrated in the embodiment as a braided member 220, which can provide characteristics to the catheter 200 such as strength, reduced kinking, wrinkling, or buckling of the shaft 202, and can provide enhanced balance for pushability, deflectability, and torque transmission such as during rotation about the longitudinal axis A. In another example, the support member 220 can include a coiled shaft member, a laser cut hypotube, the liner layer, a molded or laser cut polymer articulating joint, or other elongated shaft material. In the illustration, the braided member as the support member 220 is constructed from a woven fabric 254 or layer of braided strands or fibers 256 that form interstitial spaces 258 between the fibers 256. The braided member as the support member 220 can further be characterized by the warp and weft and bias of the fibers 256 as well as braid density such as measured in picks per inch. For instance, in some embodiments, the picks per inch can remain generally uniform along the entire longitudinal length of the braided member as the support member 220. In some embodiments, the picks per inch can vary along portions of the longitudinal length of the braided member 220. The braided member as the support member 220 can be constructed from fibers 256 that include stainless steel fibers, such as conductive fibers, or high strength polymer fibers, or as layers of different materials. In an example of a coiled member of the support member 220, the strands or fibers are wound in the same direction, and a lay direction can include clockwise or counterclockwise. Coiling can offer kink resistance and strength.

In some embodiments, the cover member 228 can be formed as a coating of reflowable plastic or thermoplastic material that extends over the support member 220, pull ring assembly 250, and liner layer and seals underlying components of the shaft 202. For instance, the coating can seep by reflowing over braided material of the support member 220 such as over the fibers 256 and into the interstitial spaces 258 of the braided material of the support member 220 and onto the liner layer. In one example, the cover member 228 is a polyether block amide and, in some examples, is available under the trade designations PEBAX from Arkema, S.A., and VESTAMID E from Evonik Industries, AG.

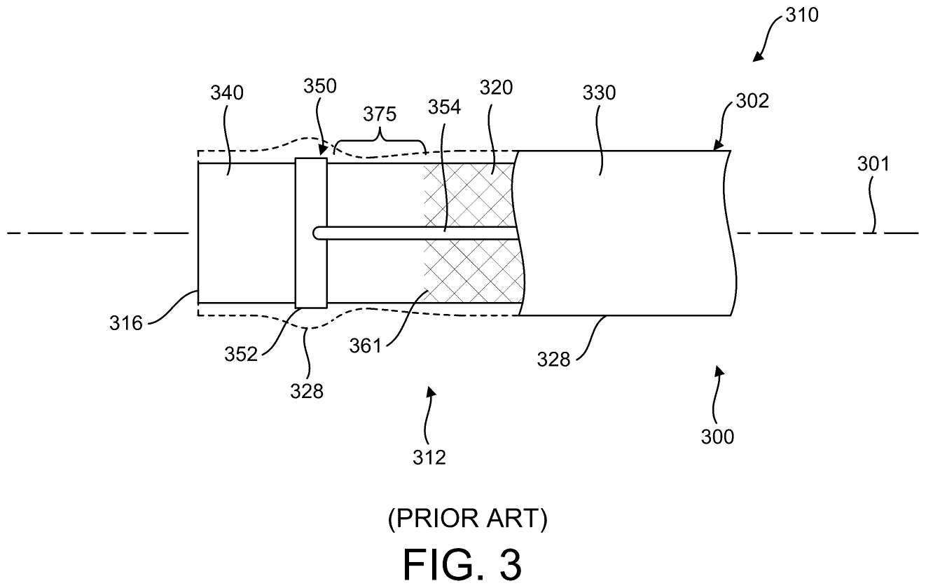

FIG. 3 illustrates a distal portion 310 of a prior art catheter 300 used in the example clinical setting 10. The catheter 300 includes an elongate shaft 302 having a distal tip section 312 and distal tip 316 on a longitudinal axis 301. The shaft 302 is constructed from several radially disposed layers such as a liner layer 340, braided support member 320 over the liner layer 340, and a cover member 328 over the support member 320 in which the cover member 328 defines an outer surface 330. The cover member 328 is shown in phantom at the distal tip section 312 and distal tip 316 for clarity. A pull ring assembly 350 is disposed over the liner layer 340 and includes an axially thin, annular pull ring 352 coupled to an elongate pull wire 354. A diametrically opposite pull wire coupled to the pull ring 352 is not shown. The pull ring 352 is axially proximal to the distal tip 316. The braided support member 320 includes a support member distal end 361 that is axially proximal to the pull ring 352. In the illustration, the pull ring 352 is distally spaced from the support member distal end by a gap 375. The cover member 328 is disposed over the support member 320 and, distal to the support member distal end 361, the cover member 328 is disposed over the pull ring assembly 350 and liner layer 340 to the distal tip 316.

Catheters 300 are difficult to manufacture with high yields. The braided support member 320 is manually cut to form the support member distal end 361 adjacent to the pull ring 352. Typically, a manufacturing technician receives two to four months of training to learn to cut the support member 320. Due to the nature of the cut support member 320, the support member distal end 361 often includes sharp ends of stainless-steel braided fibers that occasionally puncture the thin liner layer 340, which introduces leaks and compromises the lubricious liner layer 340 during manufacturing. Further, the structural axial gap 375 between the support member distal end 361 and the pull ring 342 presents a region for weakness in the steering mechanism. For instance, depending on the gap 375, the distal tip section 312 can buckle under forces without additional reinforcement. Still further, as illustrated, the outer surface 330 of the cover member 328 is prone to an uneven outside diameter of the distal portion 310 of the shaft 302 due to multi-step reflows to apply the cover member 328 over the structural gap 375.

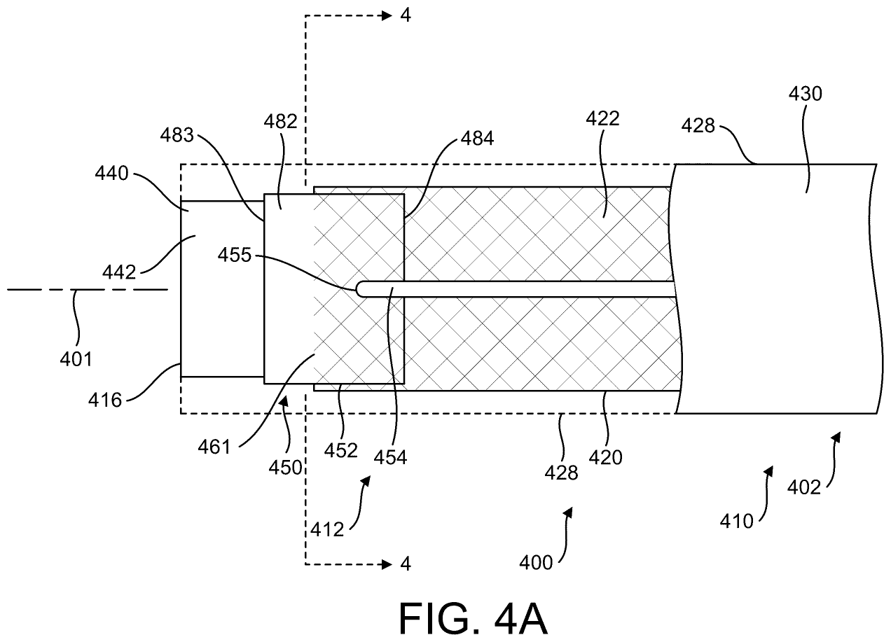

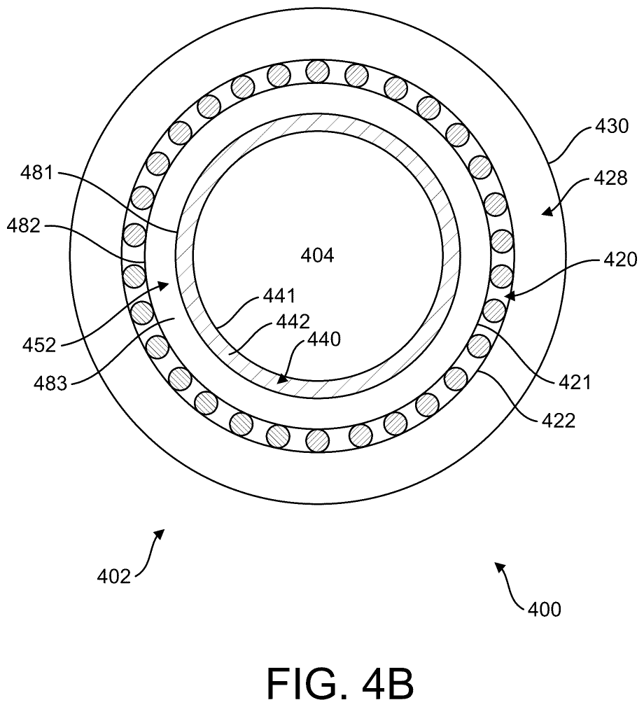

FIGS. 4A and 4B illustrate a first embodiment of a catheter 400, which is an embodiment of catheter 200 of FIG. 2 and is constructed in accordance with the present disclosure. FIG. 4A illustrates a distal portion 410 of the catheter 400, which is used in the clinical setting 10, and FIG. 4B includes a cross-section view of the embodiment of the catheter 400, such as the distal portion 410, taken along line 4-4 of FIG. 4A. The catheter 400 includes the elongated and flexible shaft 402. The shaft 402 includes a distal tip section 412 and distal tip 416 on a longitudinal axis 401. The shaft 402 includes the elongated and flexible support member 420 and cover member 428 disposed radially over the support member 420, which are coaxial with the main lumen 404. In some embodiments, the shaft 402 includes liner layer 440 also coaxial with the main lumen 404 and defining an inner wall 441 of the main lumen 404 and outer surface 442 of the liner layer 440. The support member 420 can extend longitudinally along the shaft 402 and coaxially with the main lumen 404 and disposed on the liner layer 440, such as disposed directly on the outer surface 442 of liner layer 440 as in the illustrations. The support member 420 includes an inner surface 421 disposed toward the main lumen 404, such as on the outer surface 442 of the liner layer 440, and an outer surface 422 disposed opposite inner surface 421 and the main lumen 404. In some embodiments, the shaft 402 can include a plurality of concentric or coaxially braided support members, such an innermost braided support member and an outermost braided support member. In the example, a single support member 420 is also an innermost braided support member 420. The braided support member 420 includes a support member distal end 461, which is the distalmost end of the support member 420. The cover member 428 is disposed on the outer surface 422 of the braided support member 420 and defines an outer surface 430 of the shaft 402. The cover member 428 is shown in phantom at the distal tip section 412 for clarity.

A pull ring assembly 450 is disposed over the liner layer 440, such as on the outer surface 442 of the liner layer 440 and includes an annular pull ring 452 coupled to an elongate pull wire 454. A diametrically opposite pull wire coupled to the pull ring 452 is not shown. The pull ring 452 includes an inner surface 481, an opposite outer surface 482, a distal end 483, and a proximal end 484. For example, the edges between the inner and outer surfaces 481, 482 define the distal and proximal ends 483, 484 of the annular pull ring 452. In the illustration, the inner surface 481 of the pull ring 452 is disposed on the outer surface 442 of the liner layer 440. The elongate pull wire 454 includes a pull wire distal end 455. The pull wire distal end 455 is axially (or longitudinally) disposed within the distal and proximal ends 483, 484 of the pull ring 452 and attached, such as welded, to the outer surface 482 of the pull ring 452. Accordingly, the pull wire distal end 455 of the pull wire 454 does not extend distally past the distal end 483 of the pull ring 452 in the illustrated embodiment. In the illustrated embodiment, the pull wire 454 is attached to a proximal portion of the outer surface 482 of the pull ring 452, and the pull wire distal end 455 extends distally to the proximal portion of the outer surface 482 of the pull ring 452. The pull wire 454 extends proximally underneath the support member 420, such as radially between the inner surface 421 of the support member 420 and the outer surface 442 of the liner layer 440. In the illustrated example, the pull wire 454 extends proximally and is aligned with the axis 401.

The support member 420 is attached to the pull ring 452, such as the outer surface 482 of the pull ring 452, and the support member distal end 461 is distal to the proximal end 484 of the pull ring 452. In the illustrated embodiment, the support member 420 is attached to the outer surface 482 of the pull ring 452 such that the support member distal end 461 is disposed on the outer surface 482 within the distal and proximal ends 483, 484 of the pull ring 452. Accordingly, the support member distal end 461 is not longitudinally proximal to the proximal end 484 of the pull ring 452 and, in the illustrated embodiment, does not extend longitudinally distally past the distal end 483 of the pull ring 452. Further, the pull ring 452 is radially disposed between the support member distal end 461 and the liner layer 440. For example, no part of the distal end 461 of the support member 420 is distal to the distal end 483 of the pull ring 452, and no part of the distal end 461 of the support member 420 is proximal to the proximal end 484 of the pull ring 452 such that none of the trimmed ends of the fibers contact the liner layer 440. The support member 420 is attached to the outer surface 482 of the pull ring 452, such as to a distal portion of the outer surface 482 of the pull ring 452. In the illustrated example, the support member 420 is welded to the outer surface 482 of the pull ring 452, such as to a distal portion of the outer surface 482 of the pull ring 452. In other examples, the support member 420 is bonded to the outer surface 482 of the pull ring 452, such as to a distal portion of the outer surface 482 of the pull ring 452. The support member 420 in some embodiments is attached to the outer surface 482 of the pull ring 452 via a weld or bond. In one embodiment, the support member distal end 461 is attached to the outer surface 482 of the pull ring 452.

In the illustrated embodiment of catheter 400, the liner layer 440 and the cover 428 extend distally from the pull ring assembly 450. In the illustrated embodiment, the liner layer 440 extends distally from the pull ring assembly 450, and the cover 428 is disposed directly onto the outer surface 442 of the liner layer 440 distal to the pull ring 452. In the illustrated embodiment, the cover 428 includes a cover member distal end 429 that defines the distal tip 416 of the shaft 402. The liner layer 440 includes a liner layer distal end 441, which is illustrated as extending to the distal tip 416 of the shaft 402 but can be proximal to the cover member distal end 429 and thus proximal to the shaft distal tip 416 in some embodiments.

In one embodiment, pull ring 452 is an annular ring of a selected diameter to fit over the liner layer 440 and under the support member 420 for a given catheter. Also, the longitudinal distance of the pull ring 452 between the distal and proximal ends 483, 484 is selected to be approximately within the range of 0.025 inches to 0.250 inches. The thickness of the pull ring 452 between the inner and outer surfaces 481, 482 is selected to be approximately within the range of 0.003 inches and 0.015 inches of SAE steel grade 304 stainless steel in the hardened state. Such dimensions have been determined to allow the braided support member 420 to couple to the outer surface 482 of the pull ring 452 with the support member distal end 461 longitudinally disposed between the distal and proximal ends 483, 484 without compromising the strength of the pull wire 454 weld bond to the pull ring 452. Further, the dimensions of a longer than typical pull wire ring provide for increased rigidity and a stronger bond between the outer surface of the pull ring 452 and the pull wire 454. In some examples, the pull ring includes apertures, holes, or dimples, for improving integration between the pull ring and the cover member.

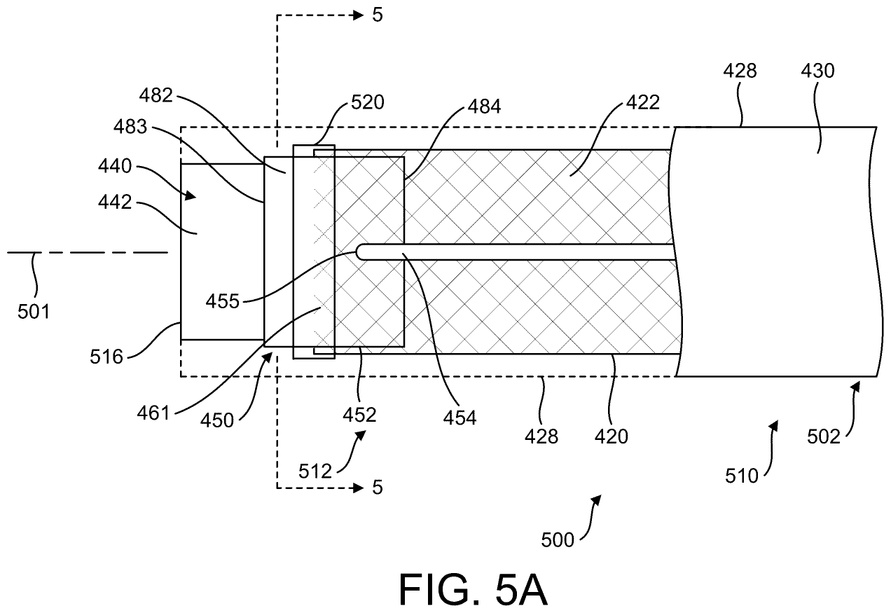

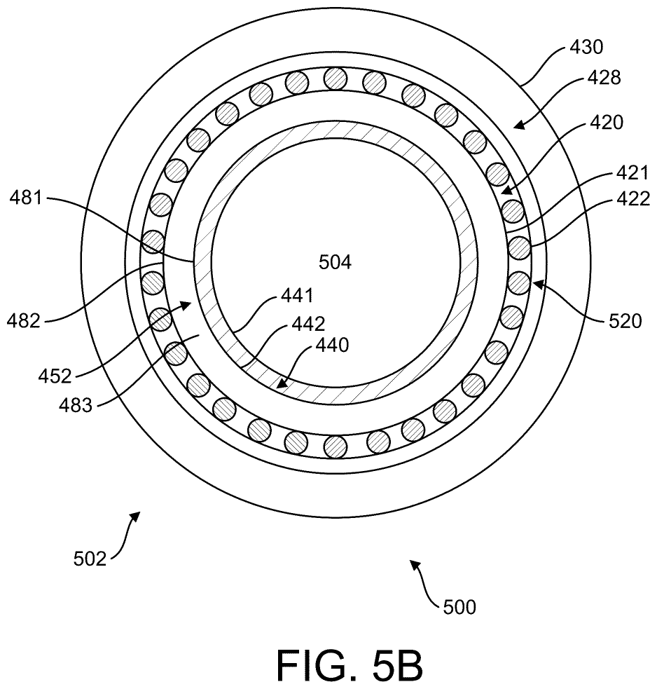

FIGS. 5A and 5B illustrate another embodiment of a catheter 500, which is an embodiment of catheter 200 of FIG. 2 and is constructed in accordance with the present disclosure. FIG. 5A illustrates a distal portion 510 of the catheter 500, which is used in the clinical setting 10, and FIG. 5B includes a cross section view of the embodiment of the catheter 500, such as the distal portion 510, taken along line 5-5 of FIG. 5A. Like parts of the catheter 500 with catheter 400, such as the liner layer 440, support member 420, cover member 428, and pull ring assembly 450, the associated features, and others, are provided with like reference numbers.

The catheter 500 includes the elongated and flexible shaft 502. The shaft 502 includes a distal tip section 512 and distal tip 516 on a longitudinal axis 501. The shaft 502 includes the elongated and flexible support member 420 and cover member 428 disposed radially over the support member 420, which are coaxial with the main lumen 504. In some embodiments, the shaft 502 includes liner layer 440 also coaxial with the main lumen 504 and defining an inner wall 441 of the main lumen 504 and outer surface 442 of the liner layer 440. The support member 420 can extend longitudinally along the shaft 402 and coaxially with the main lumen 404 and disposed on the liner layer 440, such as disposed directly on the liner layer 440 as in the illustrations. The support member 420 includes an inner surface 421 disposed toward the main lumen 504, such as on the outer surface 442 of the liner layer 440, and an outer surface 422 disposed opposite inner surface 421 and the main lumen 504. In some embodiments, the shaft 402 can include a plurality of concentric or coaxially braided support members, such an innermost braided support member and an outermost braided support member. In the example, a single support member 420 is also an innermost braided support member 420. The braided support member 420 includes a support member distal end 461, which is the distalmost end of the support member 420. The cover member 428 is disposed on the outer surface 422 of the braided support member 420 and defines an outer surface 430 of the shaft 402. The cover member 428 is shown in phantom at the distal tip section 512 for clarity.

A pull ring assembly 450 is disposed over the liner layer 440, such as on the outer surface 442 of the liner layer 440 and includes an annular pull ring 452 coupled to an elongate pull wire 454. A diametrically opposite pull wire coupled to the pull ring 452 is not shown. The pull ring 452 includes an inner surface 481, an opposite outer surface 482, a distal end 483, and an opposite proximal end 484. For example, the edges between the inner and outer surfaces 481, 482 define the distal and proximal ends 483, 484 of the annular pull ring 452. In the illustration, the inner surface 481 of the pull ring 452 is disposed on the outer surface 442 of the liner layer 440. The elongate pull wire 454 includes a pull wire distal end 455. The pull wire distal end 455 is axially (or longitudinally) aligned within the distal and proximal ends 483, 484 of the pull ring 452 and attached to the outer surface 482 of the pull ring 452. Accordingly, the pull wire distal end 455 of the pull wire 454 does not extend distally past the distal end 483 of the pull ring 452 in the illustrated embodiment. In the illustrated embodiment, the pull wire 454 is attached to a proximal portion of the outer surface 482 of the pull ring 452, and the pull wire distal end 455 extends distally to the proximal portion of the outer surface 482 of the pull ring 452. The pull wire 454 extends proximally underneath the support member 420, such as radially between the inner surface 421 of the support member 420 and the outer surface 442 of the liner layer 440.

The support member 420 is attached to the pull ring 452, such as the outer surface 482 of the pull ring 452, and the support member distal end 461 is distal to the proximal end 484 of the pull ring 452. In the illustrated embodiment, the support member 420 is attached to the outer surface 482 of the pull ring 452 such that the support member distal end 461 is disposed on the outer surface 482 within the distal and proximal ends 483, 484 of the pull ring 452. Accordingly, the support member distal end 461 is not longitudinally proximal to the proximal end 484 of the pull ring 452 and, in the illustrated embodiment, does not extend longitudinally distally past the distal end 483 of the pull ring 452. Further, the pull ring 452 is radially disposed between the support member distal end 461 and the liner layer 440. For example, no part of the distal end 461 of the support member 420 is distal to the distal end 483 of the pull ring 452, and no part of the distal end 461 of the support member 420 is proximal to the proximal end 484 of the pull ring 452 such that none of the trimmed ends of the fibers contact the liner layer 440. In the illustrated example, the support member 420 is held against to the outer surface 482 of the pull ring 452 via a compression member 520, such as to a distal portion of the outer surface 482 of the pull ring 452. The support member 420 is attached to the outer surface 482 of the pull ring 452 via compression member 520. In one embodiment, the support member distal end 461 is attached to the outer surface 482 of the pull ring 452.

The compression member 520 holds the support member 420 against the outer surface 482 of the pull ring 452 via a radial force in the direction of the axis 501. In some embodiments, the compression member 520 encircles the support member 420 and the pull ring 452 to hold the support member 420 in place against the pull ring 452. In the illustrated example, the compression member 520 encircles the distal end 461 of the support member 420 and the pull ring 452 between the distal and proximal ends 483, 484 of the pull ring 452 to hold the support member 420 in place against the pull ring 452. In one embodiment, the compression member 520 is a sleeve of a polyethylene terephthalate (PET) heat shrink material, a thermoplastic, a marker band, tape (such as a silicon tape), a metallic sleeve, or a metal ring or wire that is shrank or swaged to over the pull ring 452 to hold distal end 482 of the support member 420 in place against the pull ring 452. In one embodiment, the compression member 520 is configured to withstand a reflow process to apply the cover member 428. In some embodiments, the support member 420 can be welded to the pull ring 452 and covered with a compression member 520. Other embodiments include an undersized split ring, an slightly oversized ring minimal clearance, or an adhesive such as epoxy or a ultraviolet curable adhesive. Other embodiments are contemplated.

In the illustrated embodiment of catheter 500, the liner layer 440 and the cover 428 extend distally from the pull ring assembly 450. In the illustrated embodiment, the liner layer 440 extends distally from the pull ring assembly 450, and the cover 428 is disposed directly onto the outer surface 442 of the liner layer 440 distal to the pull ring 452. In the illustrated embodiment, the cover member distal end 429 defines the distal tip 416 of the shaft 402. The liner layer distal end 441 is illustrated as extending to the distal tip 416 of the shaft 402 but can be proximal to the cover member distal end 429 and thus proximal to the shaft distal tip 416 in some embodiments.

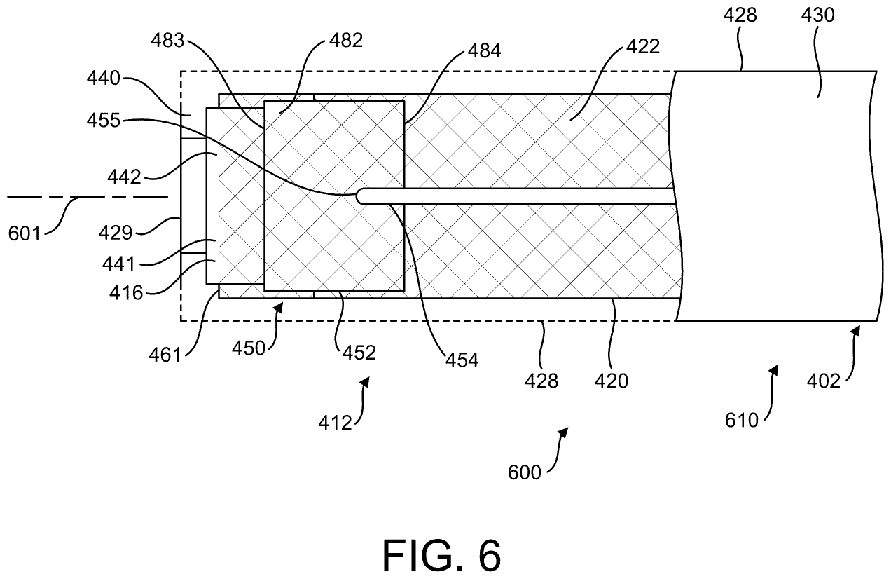

FIG. 6 illustrates another embodiment of a catheter 600, which is an embodiment of catheter 200 of FIG. 2 and is constructed in accordance with the present disclosure. FIG. 6 illustrates a distal portion 610 of the catheter 600, which is used in the clinical setting 10. Like parts of the catheter 600 with catheters 400 and 500, such as the liner layer 440, support member 420, cover member 428, and pull ring assembly 450, the associated features, and others, are provided with like reference numbers.

The catheter 600 includes the elongated and flexible shaft 402. The shaft 402 includes a distal tip section 412 and distal tip 416 on a longitudinal axis 601. The shaft 402 includes the elongated and flexible support member 420 and cover member 428 disposed radially over the support member 420, which are coaxial with the main lumen (not shown). In some embodiments, the shaft 402 includes liner layer 440 also coaxial with the main lumen 404 and defining an inner wall of the main lumen and outer surface 442 of the liner layer 440. The support member 420 can extend longitudinally along the shaft 402 and coaxially with the main lumen and disposed on the liner layer 440, such as disposed directly on the outer surface 442 of liner layer 440 as in the illustration. The support member 420 includes an inner surface (not shown) disposed toward the main lumen, such as on the outer surface 442 of the liner layer 440, and an outer surface 422 disposed opposite inner surface and the main lumen. In some embodiments, the shaft 402 can include a plurality of concentric or coaxially braided support members, such an innermost braided support member and an outermost braided support member. In the example, a single support member 420 is also an innermost braided support member 420. The braided support member 420 includes a support member distal end 461, which is the distalmost end of the support member 420. The cover member 428 is disposed on the outer surface 422 of the braided support member 420 and defines an outer surface 430 of the shaft 402. The cover member 428 is shown in phantom at the distal tip section 412 for clarity.

A pull ring assembly 450 is disposed over the liner layer 440, such as on the outer surface 442 of the liner layer 440 and includes an annular pull ring 452 coupled to an elongate pull wire 454. A diametrically opposite pull wire coupled to the pull ring 452 is not shown. The pull ring 452 includes an inner surface (not shown), an opposite outer surface 482, a distal end 483, and a proximal end 484. For example, the edges between the inner and outer surfaces 481, 482 define the distal and proximal ends 483, 484 of the annular pull ring 452. In the illustration, the inner surface of the pull ring 452 is disposed on the outer surface 442 of the liner layer 440. The elongate pull wire 454 includes a pull wire distal end 455. The pull wire distal end 455 is axially (or longitudinally) disposed within the distal and proximal ends 483, 484 of the pull ring 452 and attached, such as welded, to the outer surface 482 of the pull ring 452. Accordingly, the pull wire distal end 455 of the pull wire 454 does not extend distally past the distal end 483 of the pull ring 452 in the illustrated embodiment. In the illustrated embodiment, the pull wire 454 is attached to a proximal portion of the outer surface 482 of the pull ring 452, and the pull wire distal end 455 extends distally to the proximal portion of the outer surface 482 of the pull ring 452. The pull wire 454 extends proximally underneath the support member 420, such as radially between the inner surface of the support member 420 and the outer surface 442 of the liner layer 440. In the illustrated example, the pull wire 454 extends proximally and is aligned with the axis 601.

The support member 420 is attached to the pull ring 452, such as the outer surface 482 of the pull ring 452, and the support member distal end 461 is distal to the proximal end 484 of the pull ring 452. In the illustrated embodiment, the support member 420 is attached to the outer surface 482 of the pull ring 452 such that the support member distal end 461 is disposed longitudinally distal to the distal end 483 of the pull ring 452. Accordingly, the support member distal end 461 is not longitudinally proximal to the proximal end 484 of the pull ring 452 and, in the illustrated embodiment, does extend longitudinally distally past the distal end 483 of the pull ring 452. Further, the pull ring 452 is radially disposed between the support member 420 and the liner layer 440. For example, no part of the distal end 461 of the support member 420 is proximal to the distal end 483 of the pull ring 452 such that none of the trimmed ends of the fibers contact the liner layer 440 proximate the pull ring 452. The support member 420 is attached to the outer surface 482 of the pull ring 452, such as to the outer surface 482 of the pull ring 452. In the illustrated example, the support member 420 is welded to the outer surface 482 of the pull ring 452, such as to a distal portion of the outer surface 482 of the pull ring 452. In other examples, the support member 420 is bonded to the outer surface 482 of the pull ring 452, such as to a distal portion of the outer surface 482 of the pull ring 452. The support member 420 in some embodiments is attached to the outer surface 482 of the pull ring 452 via a weld or bond. In some embodiments, the support member 420 is held against to the outer surface 482 of the pull ring 452 via a compression member (not shown in the illustration), which can be similar to compression member 520, such as to a distal portion of the outer surface 482 of the pull ring 452. The support member 420 is attached to the outer surface 482 of the pull ring 452 via the compression member.

In the illustrated embodiment of catheter 600, the liner layer 440, the support member 420, and the cover member 428 all extend distally from the pull ring assembly 450. Accordingly, the support member distal end 461 is not longitudinally proximal to the distal end 483 of the pull ring 452. In the illustrated embodiment, the cover member distal end 429 defines the distal tip 416 of the shaft 402. The liner layer 440 includes a liner layer distal end 441, which is proximal to the cover member distal end 429 and thus proximal to the shaft distal tip 416. The support member distal end 461 is proximal to the liner layer distal end 441. Distal to the pull ring assembly 450, the cover 428 is disposed directly onto the support member 420 just distal to the pull ring assembly 450 and then directly onto the outer surface 442 of the liner layer 440 just proximate the liner layer distal end 441.

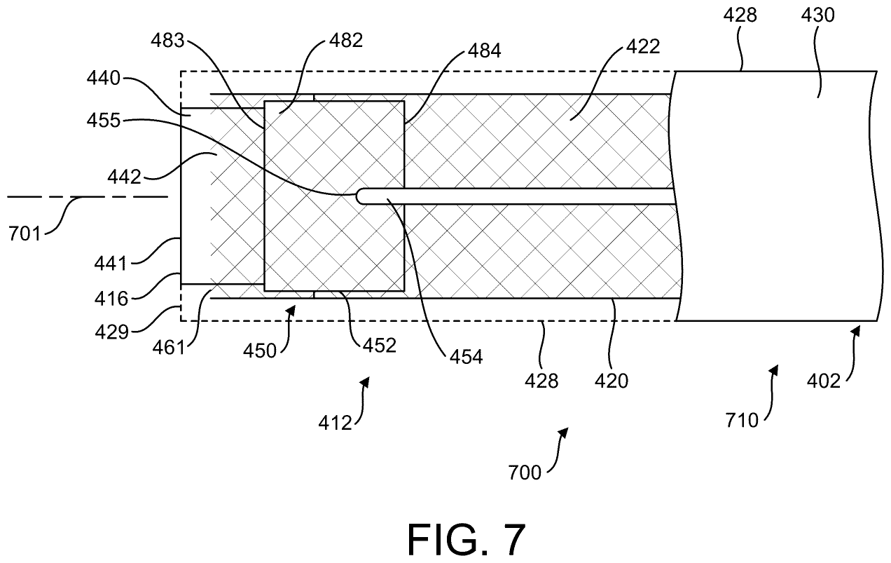

FIG. 7 illustrates another embodiment of a catheter 700, which is an embodiment of catheter 200 of FIG. 2 and is constructed in accordance with the present disclosure. FIG. 7 illustrates a distal portion 710 of the catheter 700, which is used in the clinical setting 10. Like parts of the catheter 700 with catheters 400, 500, and 600, such as the liner layer 440, support member 420, cover member 428, and pull ring assembly 450, the associated features, and others, are provided with like reference numbers.

The catheter 700 includes the elongated and flexible shaft 402. The shaft 402 includes a distal tip section 412 and distal tip 416 on a longitudinal axis 701. The shaft 402 includes the elongated and flexible support member 420 and cover member 428 disposed radially over the support member 420, which are coaxial with the main lumen (not shown). In some embodiments, the shaft 402 includes liner layer 440 also coaxial with the main lumen 404 and defining an inner wall of the main lumen and outer surface 442 of the liner layer 440. The support member 420 can extend longitudinally along the shaft 402 and coaxially with the main lumen and disposed on the liner layer 440, such as disposed directly on the outer surface 442 of liner layer 440 as in the illustration. The support member 420 includes an inner surface (not shown) disposed toward the main lumen, such as on the outer surface 442 of the liner layer 440, and an outer surface 422 disposed opposite inner surface and the main lumen. In some embodiments, the shaft 402 can include a plurality of concentric or coaxially braided support members, such an innermost braided support member and an outermost braided support member. In the example, a single support member 420 is also an innermost braided support member 420. The braided support member 420 includes a support member distal end 461, which is the distalmost end of the support member 420. The cover member 428 is disposed on the outer surface 422 of the braided support member 420 and defines an outer surface 430 of the shaft 402. The cover member 428 is shown in phantom at the distal tip section 412 for clarity.

A pull ring assembly 450 is disposed over the liner layer 440, such as on the outer surface 442 of the liner layer 440 and includes an annular pull ring 452 coupled to an elongate pull wire 454. A diametrically opposite pull wire coupled to the pull ring 452 is not shown. The pull ring 452 includes an inner surface (not shown), an opposite outer surface 482, a distal end 483, and a proximal end 484. For example, the edges between the inner and outer surfaces 481, 482 define the distal and proximal ends 483, 484 of the annular pull ring 452. In the illustration, the inner surface of the pull ring 452 is disposed on the outer surface 442 of the liner layer 440. The elongate pull wire 454 includes a pull wire distal end 455. The pull wire distal end 455 is axially (or longitudinally) disposed within the distal and proximal ends 483, 484 of the pull ring 452 and attached, such as welded, to the outer surface 482 of the pull ring 452. Accordingly, the pull wire distal end 455 of the pull wire 454 does not extend distally past the distal end 483 of the pull ring 452 in the illustrated embodiment. In the illustrated embodiment, the pull wire 454 is attached to a proximal portion of the outer surface 482 of the pull ring 452, and the pull wire distal end 455 extends distally to the proximal portion of the outer surface 482 of the pull ring 452. The pull wire 454 extends proximally underneath the support member 420, such as radially between the inner surface of the support member 420 and the outer surface 442 of the liner layer 440. In the illustrated example, the pull wire 454 extends proximally and is aligned with the axis 701.

The support member 420 is attached to the pull ring 452, such as the outer surface 482 of the pull ring 452, and the support member distal end 461 is distal to the proximal end 484 of the pull ring 452. In the illustrated embodiment, the support member 420 is attached to the outer surface 482 of the pull ring 452 such that the support member distal end 461 is disposed longitudinally distal to the distal end 483 of the pull ring 452. Accordingly, the support member distal end 461 is not longitudinally proximal to the proximal end 484 of the pull ring 452 and, in the illustrated embodiment, does extend longitudinally distally past the distal end 483 of the pull ring 452. Further, the pull ring 452 is radially disposed between the support member 420 and the liner layer 440. For example, no part of the distal end 461 of the support member 420 is proximal to the distal end 483 of the pull ring 452 such that none of the trimmed ends of the fibers contact the liner layer 440 proximate the pull ring 452. The support member 420 is attached to the outer surface 482 of the pull ring 452, such as to the outer surface 482 of the pull ring 452. In the illustrated example, the support member 420 is welded to the outer surface 482 of the pull ring 452, such as to a distal portion of the outer surface 482 of the pull ring 452. In other examples, the support member 420 is bonded to the outer surface 482 of the pull ring 452, such as to a distal portion of the outer surface 482 of the pull ring 452. The support member 420 in some embodiments is attached to the outer surface 482 of the pull ring 452 via a weld or bond. In some embodiments, the support member 420 is held against to the outer surface 482 of the pull ring 452 via a compression member (not shown in the illustration), which can be similar to compression member 520, such as to a distal portion of the outer surface 482 of the pull ring 452. The support member 420 is attached to the outer surface 482 of the pull ring 452 via the compression member.

In the illustrated embodiment of catheter 700, the liner layer 440, the support member 420, and the cover member 428 all extend distally from the pull ring assembly 450. Accordingly, the support member distal end 461 is not longitudinally proximal to the distal end 483 of the pull ring 452. In the illustrated embodiment, the cover member distal end 429 defines the distal tip 416 of the shaft 402. The liner layer distal end 441, in the illustrated embodiment, is generally co-terminal with the cover member distal end 429 and thus generally co-terminal with the shaft distal tip 416. The support member distal end 461 is proximal to the liner layer distal end 441. Distal to the pull ring assembly 450, the cover 428 is disposed directly onto the support member 420 just distal to the pull ring assembly 450 and then directly onto the outer surface 442 of the liner layer 440 just proximate the liner layer distal end 441.

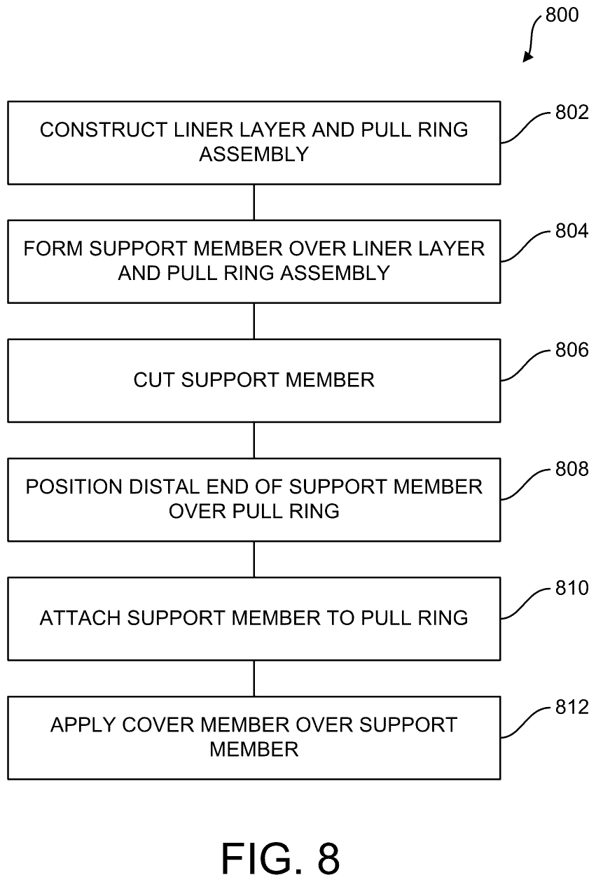

FIG. 8 illustrates an embodiment of a method 800 of manufacturing a catheter, such as catheter 200. In some embodiments, the method 800 includes constructing a partially manufactured shaft having an elongated liner layer and an exposed pull ring assembly at 802. In one embodiment, a liner layer can be formed over a mandrel and the pull wire assembly is coupled to the outer surface of the liner layer at a distal end portion of the partially manufactured shaft. In the embodiment, the distal end of a pull wire is attached to an outer surface of a pull ring to form the pull wire assembly, and the pull wire is extended along the outer surface of the liner layer.

A support member is formed for over the partially manufactured shaft at 804. In one embodiment, the support member is braided support member, and a plurality of fibers are wound or braided over the partially manufactured shaft such as from the proximal end of the shaft to the distal end of the shaft or vice versa. The braided support member is trimmed or cut over the outer surface of the pull ring or, in some embodiments, distal to the pull ring at 806. In some embodiments, the braided fibers are mechanically cut or laser cut via an automatic cutting mechanism rather than cut via a manual process to form a distal end of the support member. The distal end of the support member is positioned distal to or over the outer surface of the pull ring distal to the proximal end of the pull ring, such as between the proximal and distal ends of the pull ring, at 808. The positioned support member is attached to the outer surface of the pull ring at 810. In some embodiments, a metal or polymer tack down process is applied to attach the support member to the pull ring. In some embodiments, the support member is welded to the outer surface of the pull ring. In some embodiments, a compression member is applied over the outer surface of the support member and against the pull ring to maintain the support member against the pull ring. At 810, the pull ring is radially disposed between the liner layer and the distal end of the support member, such that the ends of the fibers do not contact the liner layer proximal to the pull ring. In some embodiments, the ends of the fibers do not contact the liner layer at all. The cover member is applied over the partially manufactured shaft including the liner layer and pull ring assembly and over the support member at 812. In one embodiment, the cover member is applied via reflow. The method 800 provides for a lower cost and timely manufacture of steerable catheters at large scale volumes, such as from increased yield, without the use of highly trained technicians manually cutting the support member.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter, comprising:
an elongated shaft defining a lumen and having a distal portion, the elongated shaft having an elongated support member coaxial with the lumen, the support member having a distal end; and
a pull ring assembly including a pull ring disposed on the shaft at the distal portion and a pull wire attached to the pull ring and extending proximally along the shaft, the pull ring having an outer surface, a pull ring distal end, and a pull ring proximal end;
wherein the support member is attached to the outer surface of the pull ring such that the distal end of the support member is disposed distal to the pull ring proximal end.

2. The catheter of claim 1, wherein the support member is attached to the outer surface of the pull ring such that the distal end of the support member is disposed between the pull ring distal and proximal ends.

3. The catheter of claim 1, wherein the support member includes a braided material forming a braided member or a coiled material forming a coiled member.

4. The catheter of claim 1, wherein the distal end of the support member is disposed distal to the pull ring distal end.

5. The catheter of claim 1, wherein the shaft includes one support member.

6. The catheter of claim 1, wherein the shaft further includes an elongate liner layer in the distal portion defining an inner wall of the lumen and having a liner layer outer surface, wherein the support member is radially disposed on the liner layer outer surface.

7. The catheter of claim 6, wherein the pull ring is radially disposed between the liner layer and the support member.

8. The catheter of claim 6, wherein the liner layer extends distally from the pull ring.

9. The catheter of claim 8, wherein the shaft includes a cover disposed on the pull ring assembly and support member.

10. The catheter of claim 9, wherein the cover is disposed directly onto the liner layer outer surface distal to the pull ring.

11. The catheter of claim 1, wherein the support member is welded to the outer surface of the pull ring.

12. The catheter of claim 1, wherein the support member is attached to the outer surface of the pull ring via a compression member.

13. The catheter of claim 12, wherein the compression member encircles the support member and the pull ring.

14. The catheter of claim 13, wherein the compression member is a sleeve of a heat-shrink material.

15. A catheter, comprising:
an elongated shaft defining a lumen and having a distal portion, the elongated shaft having an elongated support member coaxial with the lumen, the support member having a distal end; and
a pull ring assembly including a pull ring disposed on the shaft at the distal portion and a pull wire attached to the pull ring and extending proximally along the shaft, the pull ring having an outer surface, a pull ring distal end, and a pull ring proximal end;
wherein the distal end of the support member is attached to the outer surface of the pull ring.

16. The catheter of claim 15, wherein the support member includes a braided material forming a braided member or a coiled material forming a coiled member.

17. The catheter of claim 16, wherein the support member is welded or bonded to the outer surface of the pull ring.

18. A method of manufacturing a catheter, comprising:
providing an elongated shaft defining a lumen and having a distal portion, the elongated shaft having an elongated support member coaxial with the lumen, the support member having a distal end;
providing a pull ring assembly including a pull ring disposed on the shaft at the distal portion and a pull wire attached to the pull ring and extending proximally along the shaft, the pull ring having an outer surface, a pull ring distal end, and a pull ring proximal end; and
attaching the support member to the outer surface of the pull ring such that the distal end of the support member is disposed between the pull ring distal and proximal ends.

19. The method of claim 18, wherein attaching the support member to the outer surface of the pull ring includes welding or bonding the support member to the outer surface of the pull ring.

20. The method of claim 18, where attaching the support member to the outer surface of the pull ring includes encircling the support member and the pull ring with a compression member.
